(19) United States
(12) Patent Application Publication
(10) Pub. No.: US 2009/0226376 A1
(43) Pub. Date: Sep. 10, 2009

(54) Novel Mixed Ligand Core/Shell Iron Oxide Nanoparticles for Inflammation Imaging

(75) Inventors: Brian Grimmond, Clifton Park, NY (US); Brian Christopher Bales, Niskayuna, NY (US); Chiaki Treynor, Palo Alto, CA (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 12/042,701

(22) Filed: Mar. 5, 2008

(51) Int. Cl. A61K 49/18

(52) U.S. Cl. 424/9.3; 977/773

(57) ABSTRACT

A nanostructure includes (1) an inorganic nanoparticle core; (2) a first ligand, having a first chain length, bonded to the inorganic nanoparticle core; the first ligand having a charge; and (3) a second ligand, having a second chain length, bonded to the inorganic nanoparticle core; the second ligand is hydrophilic. The second chain length is longer than the first chain length such that varying a mole percent quantity of the first ligand does not substantially alter a hydrodynamic diameter of the nanostructure. Methods for making these nanostructures and their use in magnetic resonance imaging and management of inflammatory conditions are provided.

### BACKGROUND INFORMATION

A recurring challenge in developing nanoparticle-based imaging agents for magnetic resonance imaging is controlling surface charge of the MR active nanoparticles without significantly effecting the overall nanoparticle size. Nanoparticles typically have very high surface energies and as a result, they form aggregates quite easily. Nanoparticles prepared in the absence of a surface stabilizing ligands readily form aggregates in solution. This aggregation can be prevented through the binding of ligands to the surface of the nanoparticle. These ligands may prevent aggregation through either steric or electrostatic repulsions.

Typically, the surface charge of nanoparticles can be varied through the use of different stabilizing ligands that bind to the surface of the nanoparticle. Different charged stabilizing ligands are often different lengths and as a result, different ligands effect the overall size of the nanoparticle.

Known nanoparticle-based agents have included iron oxide cores stabilized by biocompatible coatings such as dextran, starch, or carbohydrate. Typically, the iron oxide core diameter ranges from about 3 to about 10 nm and the diameter of the core and coating combined ranges from about 10 to about 100 nm. Known Nanostructures, such as Feridex® and Resovist®, are negatively charged and have a short blood residence time (human blood half-life of less than 1 hour) precluding them from accessing tissue with slow uptake. Hence, agents with a short blood residence time are ill suited for imaging such tissue and subendothelial spaces, for example, the intima of blood vessels. Existing superparamagnetic particle contrast agents also suffer from various disadvantages, such as wide size distribution, agglomeration, instability, and toxicity.

Combidex®, with a dextran coating and a diameter of 15-30 nm, has been evaluated for magnetic resonance imaging in a variety of animal disease models as well as in humans. Due to its small size, Combidex® has a long blood residence time (human blood half-life between 24-36 hours.

Needs remain for nanostructures of appropriate solubility, biocompatibility, size, and coating characteristics that are capable of being efficiently internalized by inflammatory response cells and trafficked to the site of inflammation for use in imaging inflamed tissue. Given that biodistribution properties of nanoparticles designed for in vivo use are strongly influenced by the overall nanoparticle size and the surface charge, the ability to vary the nanoparticle surface charge without changing the nanoparticle size is desirable as nanoparticles of the same size with varying surface charges allow for the effects of surface charge on nanoparticle biodistribution to be decoupled from size effects.

### SUMMARY OF THE INVENTION

In some aspects, embodiments disclosed herein provide a nanostructure including: (1) an inorganic nanoparticle core; (2) a first ligand, having a first chain length, bonded to the inorganic nanoparticle core; the first ligand having a charge; and (3) a second ligand, having a second chain length, bonded to the inorganic nanoparticle core; the second ligand is hydrophilic. The second chain length is longer than the first chain length such that varying a mole percent quantity of the first ligand does not substantially alter a hydrodynamic diameter of the nanostructure.

In other aspects, embodiments disclosed herein provide a method of making the these nanostructures. The method includes (1) reacting an inorganic nanoparticle core with a first ligand having a charge. The first ligand bonds to the inorganic nanoparticle core via a functional group selected from the group consisting of a carboxylate, a sulfonate, a phosphate, and a trialkoxysilane; and (2) reacting the inorganic nanoparticle core with a hydrophilic second ligand; the second ligand bonds to the inorganic nanoparticle core via a functional group selected from a carboxylate, a sulfonate, a phosphate, and a trialkoxysilane. A molar ratio of the first ligand plus the second ligand to the inorganic nanoparticle core is between about 1:1 and about 20:1.

In yet other aspects, embodiments disclosed herein provide a method of imaging an inflammatory condition in a mammal. The method includes introducing into the mammal the above described nanostructures into inflammatory cells in vivo or ex vivo, permitting the inflammatory cells to migrate to inflamed tissue, and imaging the inflamed tissue using magnetic resonance.

Advantageously, the nanostructures disclosed herein may be useful as magnetic resonance imaging agents that can be used in visualization and management of inflammatory conditions.

The foregoing has outlined rather broadly the features of the present invention in order that the detailed description of the invention that follows may be better understood. Additional features and advantages of the invention will be described hereinafter, which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1. shows a generic structure of an magnetic resonance (MR) imaging agent having silane-linked short charged ligands L₁ and long hydrophilic ligands L₂.

FIG. 2. shows an exemplary MR imaging agent having a positively charged ligand.

FIG. 3. shows an exemplary MR imaging agent having a negatively charged ligand.

FIGS. 4A-F show T2*-weighted (A, C & E) and T1-weighted MR images before (A & B) and 24 hrs after injection of PEG-SA (C & D) and PEG-AEPTES (E & F) agents.

### DETAILED DESCRIPTION OF THE INVENTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Referring to the drawings in general, it will be understood that the illustrations are for the purpose of describing a particular embodiment of the invention and are not intended to limit the invention thereto.

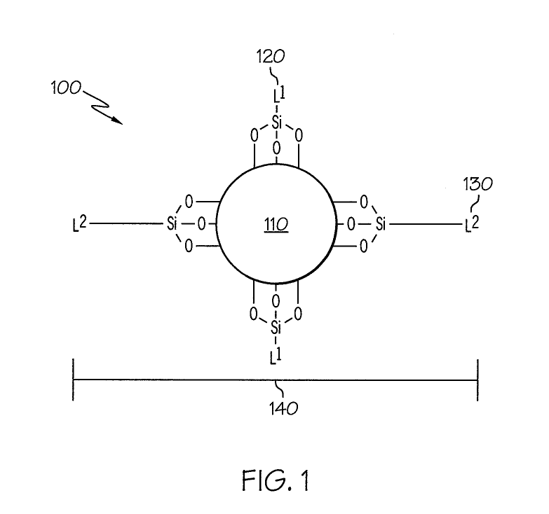

In some embodiments, the present disclosure relates to nanostructured materials that may be useful as imaging agents. With reference to FIG. 1, a nanostructure 100 for such purposes includes an inorganic nanoparticle core 110. The inorganic nanoparticle core is generally any material that can serve as a magnetic resonance (MR) active entity (i.e. a signal source for MR) and that can serve as a platform for chemical modification to affect the overall charge/size/polarity of nanostructure 100. Charge and size of nanostructure 100 are generally influenced by the ligands bound to inorganic nanoparticle core 110. These ligands minimally include a first ligand 120 and a second ligand 130. First ligand 120 has a first chain length and may be covalently bonded to inorganic nanoparticle core 110. First ligand 110 can be positively or negatively charged. Second ligand 130, has a second chain length and may be covalently bonded to inorganic nanoparticle core 110. Second ligand 130 may be hydrophilic and the second chain length is longer than the first chain length such that varying a mole percent quantity of first ligand 120 does not substantially alter the hydrodynamic diameter (D_{H}) 140 of nanostructure 100. Thus, by incorporating at least two ligands, one of sufficiently different length than the other, one may vary the amount of the charged ligand without substantially altering hydrodynamic diameter 140. Although in FIG. 1 first ligand 120 and second ligand 130 are shown to be linked via a silane moiety, the silane linkage to inorganic nanoparticle core 110 is but one exemplary embodiment as elaborated further hereinbelow.

While most of the terms used herein will be recognizable to those of skill in the art, the following definitions are nevertheless put forth to aid in the understanding of the present disclosure. It should be understood, however, that when not explicitly defined, terms should be interpreted as adopting a meaning presently accepted by those of skill in the art.

“Nanoscale,” as defined herein, generally refers to dimensions below 1 μm.

“Nanostructures,” as defined herein, generally refer to structures that are nanoscale in at least one dimension. In particular, the nanostructures disclosed herein may be useful in magnetic resonance imaging.

As used herein the terms “zeta potential,” “surface potential,” and “surface charge” and the abbreviation “ζ” refers to a measurement of the electrostatic potential near the surface of the particle. As the zeta potential is affected by the solvent and ionic strength of the solvent, all zeta potential values reported herein are measured using 10 mM aqueous NaCl as the solvent unless otherwise indicated. Thus, the cationic Nanostructures of the invention display a zeta potential of about between about 0 and about +60 mV.

As used herein “chain length” refers the longest chain of atoms of a ligand bound to an inorganic nanoparticle core.

As used herein, the terms “hydrodynamic diameter,” “hydrodynamic size,” and the abbreviation “D_{H}” refer to the diameter of spherical particle that would have a diffusion coefficient equal to that of the nanoparticle as measured by dynamic light scattering (DLS). D_{H} values may vary depending on the medium in which the agent being measured is dispersed. Thus, unless otherwise indicated, the D_{H} values described herein were measured using DLS where the agent is dispersed in 150 mM aqueous NaCl.

Inorganic nanoparticle core 110 may be any material that provides a magnetic resonance signal and is capable of chemical modification to alter the size and charge of the nanostructure. Such structures may include paramagnetic materials, superparamagnetic materials, and the like. Superparamagnetic inorganic nanoparticle cores may include (1) iron oxides (such as hematite, ferrite, and magnetite) (2) a mixed spinnel ferrite having a the general formula MFe₂O₄, where M is a metal, including without limitation, manganese, cobalt, copper, nickel, and magnesium; and (3) combinations thereof. In some embodiments, the inorganic nanoparticle core comprises a superparamagnetic iron oxide (SPIO) agent. Nanostructures may include superparamagnetic iron oxide crystalline structures that have the general formula [Fe₂⁺O₃]ₓ[Fe₂⁺O₃(M²⁺O)]₁₋ₓ where 1≧x≧0. M²⁺ may be a divalent metal ion such as iron, manganese, nickel, cobalt, magnesium, copper, or a combination thereof. When the metal ion (M²⁺) is ferrous ion (Fe²⁺) and x=0, the Nanostructure is magnetite (Fe₃O₄), and when x=1, the Nanostructure is maghemite (Fe₂O₃, γ-F₂O₃).

In general, superparamagnetism occurs when crystal-containing regions of unpaired spins are sufficiently large that they can be regarded as thermodynamically independent, single domain particles called magnetic domains. These magnetic domains display a net magnetic dipole that is larger than the sum of its individual unpaired electrons. In the absence of an applied magnetic field, all the magnetic domains are randomly oriented with no net magnetization. Application of an external magnetic field causes the dipole moments of all magnetic domains to reorient resulting in a net magnetic moment. In some embodiments, Nanostructures demonstrate a spinel crystalline structure as shown by transmission electron microscope (TEM) analysis.

The inorganic nanoparticle core may be roughly spherical in shape having a diameter ranging from about 1 nm to about 100 nm in one embodiment and from about 1 nm to about 10 nm in another embodiment. One skilled in the art will recognize that irregularities deviating from perfect spherical geometry are typical for the inorganic nanoparticle core.

In accordance with various embodiments, the ligands bound to the inorganic nanoparticle core includes at least a first ligand that carries a charge and a second ligand that is not charged, but generally hydrophilic. In some embodiments, such hydrophilic ligands should impart biocompatibility to the overall structure. Examples of chemical structures that may impart biocompatibility include, without limitation, PEG derivatives, polyvinylpyrrolidone, poly L-lysine, and the like. Biocompatibility includes solubility, generally in water, as well as non-toxicity.

The second hydrophilic ligand should have a chain length longer than the first charged ligand such that varying the amount of the charged ligand does not appreciably alter the overall size of the nanostructure, expressed herein as the hydrodynamic diameter. It is desirable to be able to vary the charge of the overall nanostructure without appreciably changing its size in order to effectively study the biodistribution of these imaging agents.

The first ligand may be bonded to the inorganic nanoparticle core by a variety of functional groups, including for example carboxylates, sulfonates, phosphates, and silanes. FIGS. 1-3 show examples of ligand linkages to an inorganic nanoparticle core via silane chemistry. Other covalent bonding motifs engaging the functional groups of a given inorganic nanoparticle core will be readily recognized by one of ordinary skill in the art. For example, with pendant OH groups on the inorganic nanoparticle core, sulfinate, sulfite, phosphinate, phosphonite, phosphonate, thiosulfate and even ether linkages are also possible. The functional group that links to the inorganic nanoparticle core and the functional end of the ligand, i.e. the charged group or the hydrophilic group may be connected via an interceding group that connects the two portions. The interceding group can vary substantially in structure, although one skilled in the art will appreciate the benefits of a minimal size for such linking group so that the overall properties of the nanostructure are not adversely affected.

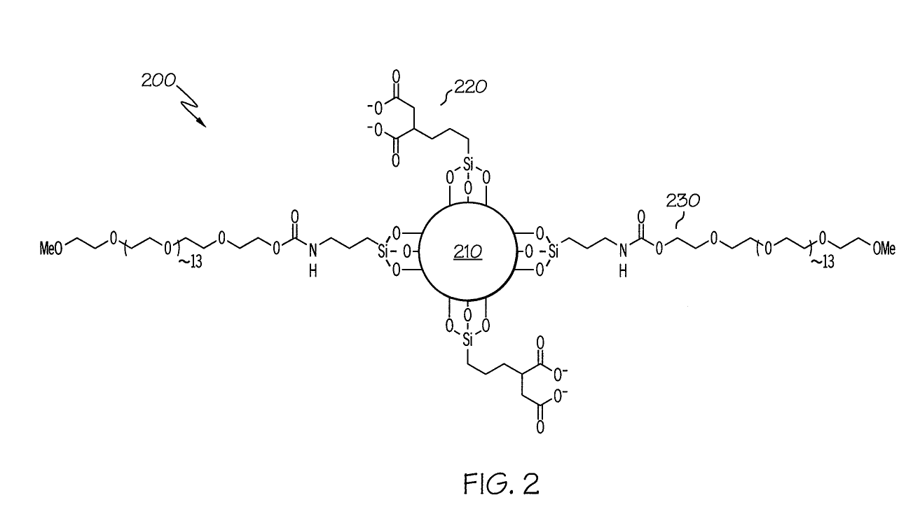

In accordance with some embodiments, the first ligand may be negatively charged. For example, a first ligand derived from the compound of formula I may be used to create nanostructure 200 shown in FIG. 2.

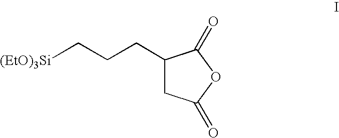

Attachment of silane I, followed by hydrolysis of the anhydride moiety provides the dicarboxylate structure of negatively charged first ligand 220. Nanostructure 200 is completed by introducing second ligand 230 bearing a PEG moiety. The ordering of attachment of the ligands may be unimportant. For example, first ligand 220 and second ligand 230 may be attached to the inorganic nanoparticle core 210 in any order or even introduced concomitantly.

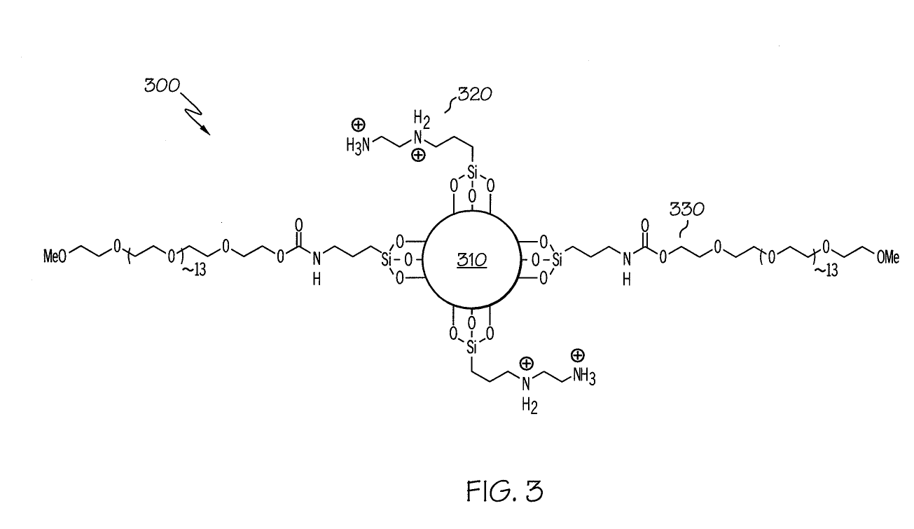

The first ligand may also be positively charged. For example a first ligand derived from the compound of formula II may be used to create nanostructure 300 shown in FIG. 3.

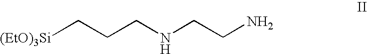

Attachment of silane II and subsequent protonation of the amino functional groups provides the positively charged first ligand 320. Nanostructure 300 is completed by introducing second ligand 330 bearing a PEG moiety. Once again, the ordering of attachment of the ligands may be unimportant. For example, first ligand 320 and second ligand 330 may be attached to the inorganic nanoparticle core 310 in any order or even introduced concomitantly.

In some embodiments the second ligand includes a PEG polymer. The PEG polymer generally has a molecular weight ranging from between about 500 and about 5000 daltons. Other homo and co-polymers that may prove useful as part of the second ligand include polyvinylpyrrolidone, poly L-lysine, and the like.

By varying the amount and charge-type of the first ligand one can introduce a non-zero surface charge on the nanostructure in a range from between about −50 mV to about +50 mV. In some embodiments, the surface charge has a non-zero surface charge in a range from between about −25 to about +25 mV. In further embodiments, the surface charge in a range from between about −5 mV to about −15 mV, and in yet further embodiments the surface charge is in a range from +5 mV to about +15 mV. One skilled in the art will recognize the value of being able to tune the surface charge depending on factors such as tissue-type being targeted, blood half-life, rate of cellular uptake, and clearance pathway.

The present disclosure provides a method of making the above described nanostructures. The method generally involves reacting an inorganic nanoparticle core with a first ligand having a charge via a carboxylate, a sulfonate, a phosphate, and a trialkoxysilane linking group and then reacting the nanoparticle core with a hydrophilic second ligand via a similar linking group (they need not be the same linking group). The order of introduction of the ligands may be inconsequential and they may even be introduced nominally simultaneously. The molar ratio of the first ligand plus the second ligand to the inorganic nanoparticle core may be in a range between about 1:1 and about 20:1. The molar ratio depends on the targeted surface potential and application for the final nanostructure. Typically, the inorganic nanoparticle core is superparamagnetic iron oxide.

When the surface charge is desirably negative, the first ligand introduced may be derived from a structure of formula I:

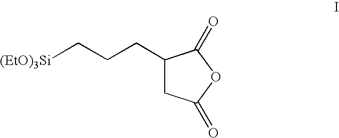

When the surface charge of the nanostructure is desirably positive, the first ligand introduced may be derived from a structure of formula II:

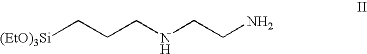

The second ligand bearing a hydrophilic group may be introduced via a structure of formula III, for example:

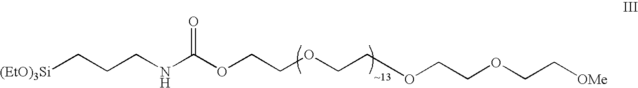

Finally, the present disclosure also provides a method of imaging an inflammatory condition in a mammal that includes introducing into the mammal (the mammal may be a human subject, for example) the nanostructures described hereinabove into inflammatory cells in vivo or ex vivo. The method includes permitting the inflammatory cells to migrate to inflamed tissue and imaging the inflamed tissue using magnetic resonance. In conjunction with the visualization methods, one can integrate management of the inflammatory condition.

The nanostructures described herein may be dispersed in physiologically acceptable carrier to minimize potential toxicity. Thus, the nanostructures of may be dispersed in a biocompatible solution with a pH of about 6 to about 8. In some embodiments, the nanostructure is dispersed in a biocompatible solution with a pH of about 7 to about 7.4. In other embodiments, the nanostructure is dispersed in a biocompatible solution with a pH of about 7.4.

The nanostructures may be combined with additives that are commonly used in the pharmaceutical industry to suspend or dissolve the compounds in an aqueous medium, and then the suspension or solution can be sterilized by techniques known in the art. The nanostructures or their pharmaceutically acceptable salts can be administered to a subject (including human subjects) in a variety of forms adapted to the chosen route of administration. Thus, the nanostructures may be introduced topically (i.e., by the administration to the tissue or mucus membranes), intravenously, intramuscularly, intradermally, and/or subcutaneously. Forms suitable for injection include sterile aqueous solutions or dispersions and sterile powders for the preparation of sterile injectable solutions, dispersions, liposomal, or emulsion formulations. In all cases, the form should be sterile and sufficiently fluid to enable administration by a syringe. Forms suitable for inhalation use include nanostructures dispersed in a sterile aerosol. Forms suitable for topical administration include creams, lotions, ointments, and the like.

In some embodiments, the nanostructures are concentrated to conveniently deliver a preferred amount of the nanostructures to a subject and packaged in container in the desired form. Thus, in some embodiments the nanostructure is dispensed in a container dispersed in physiologically acceptable solution, that conveniently facilitates administering the nanostructure in concentrations of about 0.1 mg of Fe content of the agent per kg body weight of the subject (i.e., 0.1 mg Fe/kg bw) to about 50 mg Fe/kg bw. In other embodiments, the nanostructure is packaged in a manner that conveniently facilitates administration of the nanostructure in concentrations of about 0.5 mg Fe/kg bw to about 2.5 mg Fe/kg bw.

In some embodiments, the disclosed nanostructures may be administered directly to the subject in a variety of ways including topically, intravascularly, intramuscularly, or interstitially. In some embodiments, about 0.1 mg Fe/kg to about 50 mg Fe/kg of Nanostructure is administered to the subject. In other embodiments, about 0.5 mg Fe/kg to about 2.5 mg Fe/kg of agent is administered to the subject. Similarly, inflammatory response cells containing of the disclosed nanostructures may be administered to the subject in a variety of ways including intravascularly, intramuscularly, or interstitially.

In some embodiments, the target tissue is imaged less than or approximately 3 hours after administering the nanostructures or inflammatory response cells containing the nanostructures. In alternative embodiments, the target tissue is imaged less than or approximately 24 hours after administering to the subject the nanostructures or inflammatory response cells containing nanostructures. In other embodiments, target tissue is imaged less than or approximately 5 days after administering to the subject the nanostructures or inflammatory response cells containing nanostructures.

In another series of embodiments, the present invention provides for methods of imaging conditions associated with inflammatory response cells infiltration and accumulation using the nanostructures. The nanostructures may be introduced into inflammatory response cells ex vivo and subsequently introduced into the subject. Thus, the inflammatory response cells may be withdrawn from the subject, the nanostructure introduced into the inflammatory response cells, and the inflammatory response cells containing the nanostructure are administered to subject prior to imaging. The step of introducing the nanostructures into the inflammatory response cells may optionally include the step of separating the inflammatory response cells using magnetic beads, density agents and/or centrifugation, for example. In certain embodiments, the inflammatory response cells comprise monocytes circulating in the blood, macrophage cells in tissue, dendritic cells (DCs), polynuclear monocytes (PNMs), eosinophils, neutrophils, and T cells.

The methods of managing conditions associated with inflammatory response cell infiltration and accumulation may include imaging the target tissue before, after, or both before and after treating the subject to reduce inflammation. Thus, the disclosed methods of managing conditions associated with inflammatory response cell infiltration and accumulation may include (a) imaging the target tissue to obtain base-line or diagnostic information about an inflammatory condition, (b) treating the subject, and (c) imaging the subject a one or more times to obtain further information about the inflammatory condition. A medical professional may opt not to image the subject both before and after treatment, relying on other techniques to initially characterize the inflamed tissue or subsequently assess the inflamed tissue. Thus, in an alternative embodiment, the methods of managing conditions associated with inflammatory response cell infiltration and accumulation includes treating an inflammatory condition that was identified by a technique other than magnetic resonance and imaging the target issue subsequent to treatment. Likewise, in another alternative embodiment, the disclosed methods of managing conditions associated with inflammatory response cell infiltration and accumulation may include imaging a subject or target tissue to obtain information about an inflammatory condition followed by treating the inflammatory condition without subsequently re-imaging the target tissue.

When the disease management is directed to determining the efficacy of a treatment, the methods comprise imaging the tissue of interest before administration of a treatment to obtain a pre-treatment assessment, followed by administration of the treatment and imaging the tissue of interest one or more times subsequent to the treatment to obtain a post-treatment assessment of the tissue of interest. The pre-treatment assessment and the post-treatment assessment(s) may be compared to determine whether the reduced inflammation or otherwise ameliorated the symptoms of the condition associated with inflammatory response cells infiltration and accumulation. The methods of determining the efficacy of a treatment may further comprise deciding whether to cease a particular treatment, as well as decisions to increase the frequency, intensity, and/or dose of a treatment based on the comparison of the pre- and post-treatment assessments.

When the disease management includes treatments that are localized to the inflamed tissue rather than a holistic or systemic administration of treatment (e.g., surgical or radiological intervention), the disease management methods may include determining the spatial localization of the inflamed tissue to define the specific area to be treated (e.g., excised or irradiated).

The methods described hereinabove can be used in treatments to decrease inflammation before, after, or before and after imaging the inflammatory condition. The imaging results can be used in the management of the inflammatory condition. Inflammatory conditions of particular interest are those associated with macrophage accumulation, including, without limitation, autoimmune conditions, vascular conditions, neurological conditions, and a combination thereof.

### EXPERIMENTAL EXAMPLES

The following examples are provided to more fully illustrate some of the embodiments of disclosed hereinabove. It should be appreciated by those of skill in the art that the techniques disclosed in the examples which follow represent techniques that constitute exemplary modes for its practice. However, those of skill in the art should, in light of the present disclosure, appreciate that many changes can be made in the specific embodiments that are disclosed and still obtain a like or similar result without departing from the spirit and scope of the invention.

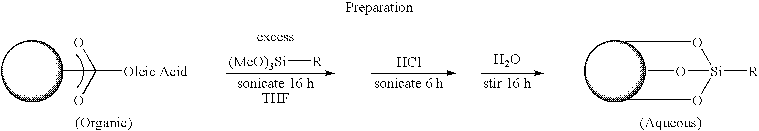

Synthesis of 5 nm SPIO nanoparticles.

A 25 mL, 3-neck Schlenk flask was fitted with a condenser, stacked on top of a 130 mm Vigreux column, and a thermocouple. The condenser was fitted with a nitrogen inlet and nitrogen flowed through the system. The Schlenk flask and Vigreux column were insulated with glass wool. Trimethylamine-N-oxide (Aldrich, 0.570 g, 7.6 mmol) and oleic acid (Aldrich: 99+%, 0.565 g, 2.0 mmol) were dispersed in 10 mL of dioctylether (Aldrich: 99%). The dispersion was heated to 80° C. at a rate of about 20° C./minutes. Once the mixture had reached ˜80° C., 265 μL of Fe(CO)₅ (Aldrich: 99.999%, 2.0 mmol) was rapidly injected into the stirring solution through the Schlenk joint. The solution turned black instantaneously, with a violent production of a white “cloud.” The solution rapidly heated to ˜120-140° C. Within 6-8 minutes the reaction pot cooled to 100° C. at which it was kept and stirred for 75 minutes. After stirring at ˜100° C. for 75 minutes, the temperature was increased to ˜280° C. at a rate of about 20° C./min. After the solution stirred for 75 minutes, the heating mantel and glass wool were removed to allow the reaction to return to room temperature.

Synthesis of PEG-750 (monomethyl ether) trimethoxy silane carbamate.

To a solution containing PEG-750 (monomethyl ether) (50.49 g, 66.0 mmol) dissolved in CH₂Cl₂ (100 mL) was added 3-isocyanatopropyltrimethoxysilane (12.54 g, 61.1 mmol) followed by dibutyl tin dilaurate (3.86 g, 6.11 mmol). The resulting solution was stirred at rt for 16 h and the solvent was removed in vacuo. The resulting residue was resuspended in MeOH (100 mL) and washed with hexanes (4×100 mL). The solvent was removed from the MeOH layer in vacuo to leave 59.3 g (100%) of the product as an off white, waxy solid. ¹H NMR (CDCl₃) δ 3.58-3.53 (m, 68H), 3.47 (s, 9H), 3.37 (s, 3H), 3.16 (m, 2H), 1.61 (m, 2H), 0.62 (m, 2H) ppm; ¹³C NMR (CDCl₃) δ 156.4, 72.5, 71.7, 70.3, 69.4, 63.5, 61.3, 58.7, 50.0, 49.6, 43.1, 22.9, 6.0 ppm; IR (neat on salt plate) 2871, 1719, 1533, 1456, 1348, 1273, 1249, 1108, 951, 821, 733, 701 cm⁻¹.

Synthesis of PEG-1900 (monomethyl ether) trimethoxy silane carbamate.

To a solution containing PEG-1900 (monomethyl ether) (20.0 g, 10.5 mmol) dissolved in CH₂Cl₂ (50 mL) was added 3-isocyanatopropyltrimethoxysilane (1.83 mL, 9.55 mmol) followed by dibutyl tin dilaurate (0.603 g, 0.955 mmol) and the resulting solution was stirred at rt for 14 d. The solvent was removed in vacuo to leave an off white solid which was dissolved in MeOH (150 mL) and washed with hexanes (4×100 mL). The MeOH was then removed in vacuo to yield 20.10 g (100%) of the product as an off white solid. ¹H NMR (CDCl₃) δ 4.21 (m, 2H), 3.54-3.82 (m, 166H), 3.49 (s, 9H), 3.31 (s, 3H), 3.16 (m, 2H), 1.62 (m, 2H), 0.64 (m, 2H); IR (neat on salt plate) 2875, 1719, 1452, 1267, 1102, 737 cm⁻¹.

Synthesis of PEG-SA (PEG-SA; 5:1) coated SPIO.

SPIO in hexadecane (11.2 mg Fe/mL) was diluted with dry THF to 1 mg Fe/mL and sonicated overnight. PEG-750 monomethyl ether trimethoxy silane carbamate (10.69 g, 10.74 mmol) was dissolved in 100 mL of above SPIO solution (100 mg Fe, 1.79 mmol). 3-(triethoxysilyl)propylsuccinic anhydride (SA) (0.601 mL, 2.11 mmol) was added to this dark, clear solution and sonicated overnight. 1M HCl (0.24 mL) was added drop wise while stirring, and mixture sonicated further for 6 hours. Water (2.0 mL) was added and the reaction was stirred at room temperature overnight. Tris/NaCl buffer (10 mM Tris, 150 mM NaCl, pH 7) was then added to the mixture (100 mL). All color was observed to transfer into the aqueous layer and the aqueous layer was washed with THF (4×100 mL). Hexanes (˜5 mL) was added as needed to facilitate phase separation. Remaining organic volatiles were removed in vacuo with gentle heating. The resulting dark aqueous solution was purified using 6 100 kDa molecular weight cutoff centrifuge filters at 4000×g by washing with 150 mM saline (4×7 mL/tube), 1:1 IPA/150 mM saline (5×7 mL/tube), and 150 mM NaCl (5×7 mL/tube). After the last wash, the dark brown aqueous solution was diluted with 150 mM NaCl to a concentration of 5 mg Fe/mL. DLS (150 mM NaCl) D_{H}=19.0 nm; zeta potential (10 mM NaCl) ζ=−10.0 mV.

Synthesis of PEG-SA (PEG-SA; 2:1) coated SPIO.

SPIO in hexadecane (11.2 mg Fe/mL) was diluted with dry THF to 1 mg Fe/mL and sonicated overnight. PEG-750 monomethyl ether trimethoxy silane carbamate (8.52 g, 8.78; mmol) was dissolved in 100 mL of above SPIO solution (100 mg Fe, 1.79 mmol). 3-(triethoxysilyl)propylsuccinic anhydride (SA) (1.31 mL, 4.33 mmol) was added to this dark, clear solution and sonicated overnight. 1M HCl (0.24 mL) was added drop wise while stirring, and mixture sonicated further for 6 hours. Water (2.0 mL) was added and the reaction was stirred at room temperature overnight. Phosphate buffered saline (PBS) (154 mM NaCl, 10 mM sodium phosphate, pH=7.4) was then added to the mixture (100 mL). All color was observed to transfer into the aqueous layer and the aqueous layer was washed with THF (4×100 mL). Hexanes (˜5 mL) was added as needed to facilitate phase separation. Remaining organic volatiles were removed in vacuo with gentle heating. The resulting dark aqueous solution was purified using 6 100 kDa molecular weight cutoff centrifuge filters at 4000×g by washing with water (1×7 mL/tube). The resulting solution was adjusted to pH 10 with NH₄OH and stirred at rt for 3d. The resulting dark brown aqueous solution was purified using 6 100 kDa molecular weight cutoff centrifuge filters at 4000×g by washing with 1:1 IPA/PBS (5×7 mL/tube), and PBS (5×7 mL/tube). After the last wash, the dark brown aqueous solution was diluted with PBS to a concentration of 5 mg Fe/mL. DLS (PBS) D_{H}=20.1 nm; zeta potential (10 mM NaCl) ζ=−10.6 mV.

Synthesis of PEG-SA (PEG-SA; 1:1) coated SPIO.

SPIO in hexadecane (11.2 mg Fe/mL) was diluted with dry THF to 1 mg Fe/mL and sonicated overnight. PEG-750 monomethyl ether trimethoxy silane carbamate (6.36 g, 6.56 mmol) was dissolved in 100 mL of above SPIO solution (100 mg Fe, 1.79 mmol). 3-(triethoxysilyl)propylsuccinic anhydride (SA) (2.00 mL, 6.56 mmol) was added to this dark, clear solution and sonicated overnight. 1M HCl (0.24 mL) was added drop wise while stirring, and mixture sonicated further for 6 hours. Water (2.0 mL) was added and the reaction was stirred at room temperature overnight. Phosphate buffered saline (PBS) (154 mM NaCl, 10 mM sodium phosphate, pH=7.4) was then added to the mixture (100 mL). All color was observed to transfer into the aqueous layer and the aqueous layer was washed with THF (4×100 mL). Hexanes (˜5 mL) was added as needed to facilitate phase separation. Remaining organic volatiles were removed in vacuo with gentle heating. The resulting dark aqueous solution was purified using 6 100 kDa molecular weight cutoff centrifuge filters at 4000×g by washing with water (1×7 mL/tube). The resulting solution was adjusted to pH 10 with NH₄OH and stirred at rt for 3d. The resulting dark brown aqueous solution was purified using a 100 kDa molecular weight cutoff centrifuge filter at 4000×G by washing with 1:1 IPA/PBS (5×7 mL/tube), and PBS (5×7 mL/tube). After the last wash, the dark brown aqueous solution was diluted with PBS to a concentration of ˜5 mg Fe/mL. DLS (PBS) D_{H}=19.4 nm; zeta potential (10 mM NaCl) ζ=−30.5 mV.

Synthesis of PEG-AEAPTES (PEG-AEAPTES; 5:1) coated SPIO

SPIO in hexadecane (11.2 mg Fe/mL) was diluted with anhydrous tetrahydrofuran to a concentration of 1 mg Fe/mL and this solution was sonicated overnight in a VWR 150T model sonicator. PEG750 monomethylether trimethoxysilane carbamate (15.9 g, 16.1 mmol) was dissolved in the SPIO solution (150 mL, 2.68 mmol), N-(2-aminoethyl)-3-aminopropyltriethoxysilane (0.86 mL, 3.22 mmol) was added and the dark mixture was sonicated for 17 h. 1.2 M HCl (0.3 mL) was added and the mixture was sonicated for an additional 9 h. Water (3 mL, 2% (v/v)) was added and the reaction mixture was stirred at room temperature for 15 h, then quenched with a 10 mM Tris/150 mM NaCl aqueous solution (150 mL). The layers were separated and the dark aqueous layer was extracted with 5% hexanes in tetrahydrofuran (6×100 mL). The remaining aqueous mixture was further concentrated under reduced pressure at 30° C. for 1 h. The SPIO solution was purified on 100 kDa molecular weight cutoff filters at 4000×g by washing with sterile 150 mM NaCl (4×), 1:1 isopropanol: 150 mM NaCl (4×) and sterile 150 mM NaCl (5×). The remaining concentrated solution was diluted with sterile 150 mM NaCl (pH 3) and the iron concentration was determined by UV absorption using Perl's reagent (aq K₄Fe(CN)₆; 50 mg/mL). Final concentration was targeted to be 10 mg Fe/mL: D_{H} (150 mM NaCl, pH 3)=18.2 nm; Zeta potential (10 mM NaCl, pH 7) ζ=10.6 mV.

Synthesis of PEG-AEAPTES (PEG-AEAPTES; 2.5:1) coated SPIO

SPIO in hexadecane (11.2 mg Fe/mL) was diluted with anhydrous tetrahydrofuran to a concentration of 1 mg Fe/mL and this solution was sonicated for 13 h in a VWR 150T model sonicator. PEG750 monomethylether trimethoxysilane carbamate (10.6 g, 10.7 mmol) was dissolved in the SPIO solution (100 mL, 1.79 mmol), N-(2-aminoethyl)-3-aminopropyltriethoxysilane (1.14 mL, 4.30 mmol) was added and the dark mixture was sonicated for 16 h. 1.2 M HCl (0.2 mL) was added and the mixture was sonicated for an additional 24 h. Water (2 mL, 2% (v/v)) was added and the reaction mixture was stirred at room temperature for 24 h, then quenched with a 10 mM Tris/150 mM NaCl aqueous solution (100 mL). The layers were separated and the dark aqueous layer was extracted with 5% hexanes in tetrahydrofuran (5×100 mL). The remaining aqueous mixture was further concentrated under reduced pressure at 40° C. for 15 min. The SPIO solution was purified on 100 kDa molecular weight cutoff filters at 4000×g by washing with sterile 150 mM NaCl (4×), 1:1 isopropanol:150 mM NaCl (4×) and sterile 150 mM NaCl (5×). The remaining concentrated solution was diluted with sterile 150 mM NaCl (pH 3) and the iron concentration was determined by UV absorption using Perl's reagent (aq K₄Fe(CN)₆; 50 mg/mL). Final concentration was targeted to be 10 mg Fe/mL: D_{H} (150 mM NaCl, pH 3)=21.9 nm; Zeta potential (10 mM NaCl, pH 7) ζ=19.74 mV.

Synthesis of PEG-AEAPTES (PEG-AEAPTES; 10:1) coated SPIO

SPIO in hexadecane (11.2 mg Fe/mL) was diluted with anhydrous tetrahydrofuran to a concentration of 1 mg Fe/mL and this solution was sonicated for 15 h in a VWR 150T model sonicator. PEG750 monomethylether trimethoxysilane carbamate (11.2 g, 11.4 mmol) was dissolved in the SPIO solution (106 mL, 1.90 mmol), N-(2-aminoethyl)-3-aminopropyltriethoxysilane (0.3 mL, 1.14 mmol) was added and the dark mixture was sonicated for 17 h. 1.2 M HCl (0.212 mL) was added and the mixture was sonicated for an additional 8 h. Water (2.1 mL, 2% (v/v)) was added and the reaction mixture was stirred at room temperature for 17 h, then quenched with a 10 mM Tris/150 mM NaCl aqueous solution (100 mL). The layers were separated and the dark aqueous layer was extracted with 5% hexanes in tetrahydrofuran (5×100 mL). The remaining aqueous mixture was further concentrated under reduced pressure at 40° C. for 15 min. The SPIO solution was purified on 100 kDa molecular weight cutoff filters at 4000×g by washing with sterile 150 mM NaCl (4×), 1:1 isopropanol:150 mM NaCl (4×) and sterile 150 mM NaCl (5×). The remaining concentrated solution was diluted with sterile 150 mM NaCl (pH 3) and the iron concentration was determined by UV absorption using Perl's reagent (aq K₄Fe(CN)₆; 50 mg/mL). Final concentration was targeted to be 10 mg Fe/mL: D_{H} (150 mM NaCl, pH 3)=21.8 nm; Zeta potential (10 mM NaCl, pH 7) ζ=3.39 mV.

Synthesis of PEG-tetramethyl ammonium (PEG-Ammonium; 5:1) coated SPIO

SPIO in hexadecane (11.2 mg Fe/mL) was diluted with anhydrous tetrahydrofuran to a concentration of 1 mg Fe/mL and this solution was sonicated for 13 h in a VWR 150T model sonicator. PEG750 monomethylether trimethoxysilane carbamate (7.42 g, 7.52 mmol) was dissolved in the SPIO solution (70 mL, 1.25 mmol), N-trimethoxysilylpropyl-N,N,N-trimethyllammonium chloride (50% solution in methanol, 0.83 mL, 1.50 mmol) was added and the dark mixture was sonicated for 16 h. 1.2 M HCl (0.14 mL) was added and the mixture was sonicated for an additional 24 h. Water (1.4 mL, 2% (v/v)) was added and the reaction mixture was stirred at room temperature for 24 h, then quenched with a 10 mM Tris/150 mM NaCl aqueous solution (72 mL). The layers were separated and the dark aqueous layer was extracted with 5% hexanes in tetrahydrofuran (4×100 mL). The remaining aqueous mixture was further concentrated under reduced pressure at 40° C. for 15 min. The SPIO solution was purified on 100 kDa molecular weight cutoff filters at 4000×g by washing with sterile 150 mM NaCl (4×), 1:1 isopropanol:150 mM NaCl (4×) and sterile 150 mM NaCl (5×). The remaining concentrated solution was diluted with sterile 150 mM NaCl (pH 3) and the iron concentration was determined by UV absorption using Perl's reagent (aq K₄Fe(CN)₆; 50 mg/mL). Final concentration was targeted to be 10 mg Fe/mL: D_{H} (150 mM NaCl, pH 3)=29.6 nm; Zeta potential (10 mM NaCl, pH 7) ζ=18.41 mV.

Synthesis of PEG₁₉₀₀-AEAPTES (PEG₁₉₀₀-AEAPTES; 5:1) coated SPIO.

SPIO in hexadecane (11.2 mg Fe/mL) was diluted with dry THF to 1 mg Fe/mL and sonicated overnight. PEG-1900 monomethyl ether trimethoxy silane carbamate (2.31 g, 1.10 mmol) was dissolved in 10 mL of above SPIO solution (10 mg Fe, 0.179 mmol). N-(2-aminoethyl)-3-aminopropyltriethoxysilane (AEAPTES) (0.057 mL, 0.214 mmol) was added to this dark, clear solution and sonicated overnight. 1M HCl (0.024 mL) was added drop wise while stirring, and mixture sonicated further for 6 hours. Water (0.200 mL) was added and the reaction was stirred at room temperature overnight. Tris/NaCl buffer (10 mM Tris, 150 mM NaCl, pH ˜7) was then added to the mixture (10 mL). All color was observed to transfer into the aqueous layer and the aqueous layer was washed with THF (4×10 mL). Hexanes (˜5 mL) was added as needed to facilitate phase separation. Remaining organic volatiles were removed in vacuo with gentle heating. The resulting dark aqueous solution was purified using a 100 kDa molecular weight cutoff centrifuge filter at 4000×g by washing with 150 mM saline (4×7 mL/tube), 1:1 IPA/150 mM saline (5×7 mL/tube), and 150 mM NaCl (5×7 mL/tube). After the last wash, the dark brown aqueous solution was diluted with 150 mM NaCl, pH 3 to a concentration of ˜5 mg Fe/mL. DLS (150 mM NaCl, pH 3) D_{H}=27.8 nm; zeta potential (10 mM NaCl) ζ=7.9 mV.

Characterization

Hydrodynamic diameter was measured via dynamic light scattering using 150 mM NaCl as the solvent. The purified SPIO solution was diluted with 150 mM NaCl and passed through a 100 nm filter prior to DLS analysis using a Brookhaven ZetaPALS. Zeta potential was measured using a Brookhaven ZetaPALS after diluting the SPIO solution 14× with H₂O (final solution (10 mM NaCl) and passing the diluted SPIO solution through a 100 nm filter. The results are shown below in Table 1.

TABLE 1

| | | | | Surface |
|---|---|---|---|---|
| | | mol % | | Charge |
| | Charged | Charged | Size | (zeta |
| Example | Ligand* | Ligand | (DH) | potential) |
| 1 | AEAPTES | 50% | 18.6 | +33 mV |
| | | | nm | |
| 2 | AEAPTES | 29% | 17.5 | +26 mV |
| | | | nm | |
| 3 | AEAPTES | 17% | 19.4 | +11 mV |
| | | | nm | |
| 4 | SA | 20% | 22.2 | −10 mV |
| | | | nm | |
| 5 | SA | 33% | 20.1 | −11 mV |
| | | | nm | |
| 6 | SA | 50% | 19.4 | −30 mV |
| | | | nm | |

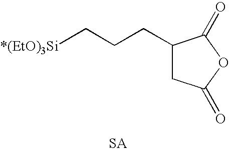

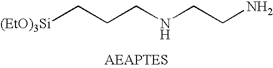

In vivo experiments

Granulomas were induced in female Swiss Webster mice by subcutaneous injection of 0.1 mL of a 1% carrageenan suspended in sterile physiologic phosphate-buffered saline. The injection site was dorsally located ˜1 cm superior to the base of tail. SPIO contrast agent was then injected intravenously via the tail vein in physically restrained mice between 2 and 7 days following granuloma induction. SPIO agent was in physiologic saline at a concentration of 5 mg Fe/mL, and was sterile filtered prior to injection and tested for the presence of endotoxin. The agent was dosed at 20 mg Fe/kg body weight.

The mice were imaged prior to injection of SPIO contrast agent, and again at ˜24 hrs post injection of the agent. Mice were imaged on a clinical 1.5 T GE Signa MR scanner using a custom-built, 3.2 cm solenoid transmit/receive RF coil. The mice were anesthetized using 2% isoflurane in oxygen by nose cone using a commercial anesthesia machine designed for rodents. For each of 2 pulse sequences, 13 transaxial 1 mm image slices were collected to obtain full coverage of the granuloma. The pulse sequence parameters were as follows:
T1-weighting: 2D Spin Echo, TE 13, TR 320, matrix 256×192, FOV 5, phase FOV 0.75, thickness 1.0, NEX 3, BW 22.73.
T2*-weighting: 2D Gradient Echo, TE1 9.8, TE2 25, TR 650, flip ang 45, matrix 256×192, FOV 5, phase FOV 0.75, slice thickness 1.0, NEX 2, BW 15.63.

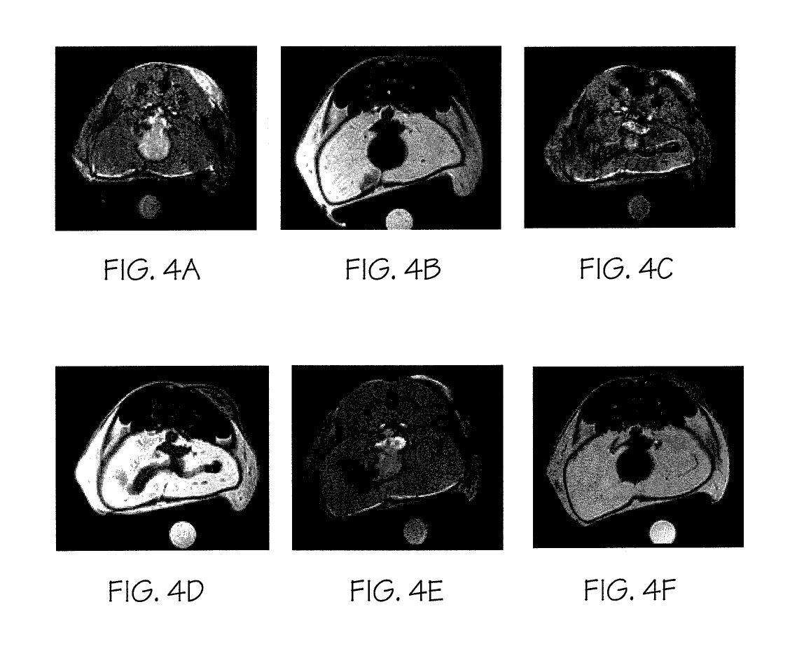

FIGS. 4A-F show T2*-weighted (A, C & E) and T1-weighted MR images before (A & B) and 24 hrs after injection of PEG-SA (C & D) and PEG-AEPTES (E & F) agents. Granuloma location is indicated by arrows. Notable T2* (dark) and T1 (bright) contrast is observed within the granuloma, exhibiting the ability of the SPIO agent to provide contrast in inflammatory lesions.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A nanostructure comprising:
an inorganic nanoparticle core;
a first ligand having a first chain length, bonded to the inorganic nanoparticle core;
wherein the first ligand is charged; and
a second ligand, having a second chain length, bonded to the inorganic nanoparticle core;
wherein the second ligand is hydrophilic; and
wherein the second chain length is longer than the first chain length such that varying a mole percent quantity of the first ligand does not substantially alter a hydrodynamic diameter of the nanostructure.

2. The nanostructure of claim 1, wherein the inorganic nanoparticle core comprises superparamagnetic iron oxide.

3. The nanostructure of claim 1, wherein the inorganic nanoparticle core has a diameter ranging from about 1 nm to about 100 nm.

4. The nanostructure of claim 1, wherein the inorganic nanoparticle core has a diameter of about 1 nm to about 10 nm.

5. The nanostructure of claim 1 having a hydrodynamic diameter of about 1 nm to about 500 nm.

6. The nanostructure of claim 1 having a hydrodynamic diameter of about 1 nm to about 100 nm.

7. The nanostructure of claim 1 having a hydrodynamic diameter of about 2 nm to about 30 nm.

5. The nanostructure of claim 1, wherein the first ligand and second ligand bond to the inorganic nanoparticle core by a functional group selected from a carboxylate, a sulfonate, a phosphate, and a silane and mixtures thereof.

6. The nanostructure of claim 1, wherein the first ligand is negatively charged.

7. The nanostructure of claim 6, wherein the first ligand is derived from a structure of formula I:

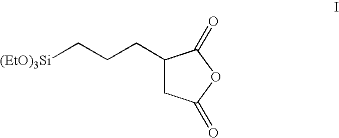

8. The nanostructure of claim 1, wherein the first ligand is positively charged.

9. The nanostructure of claim 8, wherein the first ligand is derived from a structure of formula II:

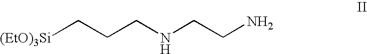

10. The nanostructure of claim 1, wherein the second ligand comprises a PEG polymer.

11. The nanostructure of claim 10, wherein the PEG polymer has a molecular weight ranging from between about 500 and 5000 daltons.

12. The nanostructure of claim 1 having a non-zero zeta potential in a range from between about −50 mV to about +50 mV.

13. The nanostructure of claim 12 having a non-zero zeta potential in a range from between about −25 to about +25 mV.

14. The nanostructure of claim 13 having a zeta potential in a range from between about −5 mV to about −15 mV.

15. The nanostructure of claim 13 having a zeta potential in a range from between about +5 mV to about +15 mV.

16. A method of making the nanostructure of claim 1 comprising:
reacting an inorganic nanoparticle core with a first ligand having a charge;
wherein the first ligand bonds to the nanoparticle core via a functional group selected from the group consisting of a carboxylate, a sulfonate, a phosphate, and a trialkoxysilane; and
reacting the nanoparticle core with a hydrophilic second ligand;
wherein the second ligand bonds to the nanoparticle core via a functional group selected from a carboxylate, a sulfonate, a phosphate, and a trialkoxysilane;
wherein a molar ratio of the first ligand plus the second ligand to the inorganic nanoparticle core is between about 1:1 and about 20:1.

17. The method of claim 16, wherein the inorganic nanoparticle core is superparamagnetic iron oxide.

18. The method of claim 16, wherein the first ligand is derived from a structure of formula I:

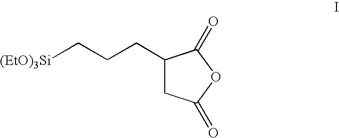

19. The method of claim 16, where in the first ligand is derived from a structure of formula II:

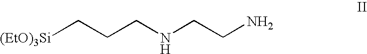

20. The method of claim 16, wherein the second ligand is derived from a structure of formula III:

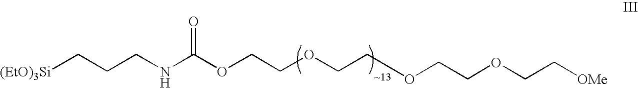

21. A method of imaging an inflammatory condition in a mammal comprising
introducing into the mammal the nanostructure of claim 1;
permitting the nanostructure of claim 1 to migrate to inflamed tissue; and
imaging the inflamed tissue using magnetic resonance.

22. The method of claim 21 further comprising managing the inflammatory condition.

23. The method of claim 21 wherein the mammal is a human.

24. The method of claim 21, further comprising treating the mammal to decrease inflammation before, after, or before and after imaging the inflammatory condition, and using the results to manage the inflammatory condition.

25. The method of claim 21, wherein the introducing step comprises administering the agent topically, intravascularly, intramuscularly, or interstitially.

26. The method of claim 23, wherein about 0.1 mg Fe/kg to about 50 mg Fe/kg of the nanostructure is administered to the human.

27. The method of claim 23, wherein about 0.1 mg Fe/kg to about 2.5 mg Fe/kg of the nanostructure is administered to the human.

28. The method of claim 21, wherein the inflammatory condition is associated with macrophage accumulation.

29. The method of claim 21, wherein the inflammatory condition is a condition selected from the group consisting of an autoimmune condition, a vascular condition, a neurological condition, and a combination thereof.
